(19) United States
(12) Patent Application Publication
(10) Pub. No.: US 2009/0099548 A1
(43) Pub. Date: Apr. 16, 2009

(54) Device for Regenerating Adipose Tissue and Method for Utilizing Thereof

(76) Inventors: Hyun-Mi Son, Seoul (KR); Jae-Deog Jang, Seoul (KR); Cheong-Ho Chang, Seoul (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 12/224,738

(22) PCT Filed: Mar. 15, 2006
(86) PCT No.: PCT/KR2006/000924

§ 371 Date: Sep. 3, 2008

(30) Foreign Application Priority Data
Mar. 8, 2006 (KR) 10-2006-0021839

(51) Int. Cl. A61K 35/12; A61M 5/31

(52) U.S. Cl. 604/522; 604/60; 424/574

(57) ABSTRACT

A device and method for regenerating adipose tissue has developed comprising: a washing container filled with an sterile solution for washing the adipose tissues; a syringe container holding syringes for washing and separating adipose tissues; injecting the wash solution and mixing the Bio-Gel, respectively; a second syringe container containing Bio-Gel syringes filled with materials to induce adipogenesis; and connector containers holding connectors for injection of the wash solution, and mixing of washed tissues and Bio-Gel. These containers are restrained in the kit by grooves in the bottom of the box. The box top holds needle containers for transplantation needles; and a protocol for fat transplantation, restrained by grooves. The kit minimizes risk of infection in fat transplantations using the patient's own tissues. It enables stable tissue transplantation as well as adipose tissue regeneration via use of aseptic/sterile implements, and a protocol for fat transplantation, solving the problems associated with conventional adipose tissue transplantation.

### BACKGROUND OF THE INVENTION

1. Field of the Invention

The present invention relates to a device for regenerating adipose tissue and a method thereof. More specifically, the device forming a set of single kit and the method for regenerating the adipose tissue minimize the risk of infection in fat transplantation during the treatment and reconstruction of defect regions by using patient's own adipose tissues that are transplantable into patients. Those are suitable to perform the stable surgical operation of adipose tissue transplantation, as well as induction of adipose tissue regeneration via constitution of aseptic/sterile implements and a standardized protocol of fat transplantation.

2. Related Prior Art

According to Melvin A. Shiffman in “Autologous fat transplantation”, the first clinical fat transplantation was performed by Neuber. In his medical paper published in 1893, Neuber conducted fat transplantation with some satisfactory results. He collected the almond-sized piece of fat as a filling material, and transplanted it to fill in a depressed area of the face which had resulted from infection of tuberculosis into bones.

Since then, many cases of fat injection under various situations have been attempted by a great number of people. However, all these early attempts led to disappointing results. For example, in 1895, Czerny attempted fat injection to fill a breast defect created by removal of a benign breast tumor. In an article published in 1909, Verderame introduced fat injection into an oculoplastic surgery. However, since most of fat thus injected was absorbed by the body, it was also recommended to inject greater amounts of fat than necessary.

Over the almost one hundred years since then, a variety of attempts of fat injection have been conducted, but the results exhibited significant variation and differences among surgeons who have published the results of their research. Therefore, these results have raised disappointment and caused many surgeons to give up, and further, fat injection techniques were confronted with the urgency of being kept secret. However, as numerous methods of reducing body's absorption of fat following fat injection have recently been developed, fat injection techniques have again been receiving a great deal of attention.

Upon scrutinizing various medical articles dealing with fat injection techniques, early attempts and results show disappointing outcomes, with high absorption rates of more than 50% within a year. Every surgeon who has conducted fat transplantation points out a variety of factors that interfere with engrafting of transplanted fat and the body's increased absorption of fat. For instance, various suggestions have been continuously made on methods of reducing body's absorption of fat in order to increase the engraft rate of lipoinjection, and on various methods for enhancing engrafting of fat, including the following various experimental results: differences in engraft rate depending on from where the fat is collected; differences in an engraft rate according to collection methods of the fat; experiments to find proper pressure for liposuction; whether washing of collected fat is preferable or not; differences in an engraft rate depending upon injection methods, injection volume or injection mode including a bulk injection or divided injection of fat; and an increased engraft rate by fat injection in combination with addition of betamethasone, insulin or dextran.

In 1928, Hilse histologically confirmed formation of adipose tissues from free fat grafts without exception and called histocytes filled with fat, “lipoblasts”. In 1947, Green treated bone defects secondary to osteomyelitis using fat, and considered that transplanted fat will become connective tissues and finally undergo ossification, thus being capable of filling in defect regions. In 1956, Peer published his work with autogenous dermal fat grafts. In his work, he cut a volume of fat corresponding to walnut-sized mass into 20 pieces and transplanted it into the defect area. Upon examining the transplanted fat several times during one year, he found that transplantation of bulk fat exhibited a high engraft rate as compared to transplantation of fat in small pieces. To the contrary, Coleman has asserted, through his article published in 1995, that when fat is divided into several layers and injected little by little, there are more opportunities for supply of oxygen and nutrients and thus it is preferred that fat was proportionally injected in the form of a multilayer.

Further, as mechanical liposuction using cannula was made possible by A. Fisher and G. Fisher in 1974, Bircoll has made an effort to employ the liposuctioned fat in lipoinjection in 1982 and such a method has been in use since then.

Meanwhile, regarding autologous fat transplantation, the American Society of Plastic and Reconstruction Surgery (ASPRS) Ad-Hoc Committee on New Procedures (1987) states as follows: autologous fat injection should have historical and scientific basis and is still experimental surgery, and therefore the results of fat injection are too various. As a result, in order to make a conclusion on whether fat injection is useful or not, controlled clinical studies are required.

Conventional autologous fat transplantation, as discussed above, is still prevalently performed by surgeons in many hospitals and is a transplantation technique for reconstruction of soft tissue defects which is in on-going development. However, throughout the world until now, there is no case of combined provision of easy manipulation method for fat transplantation, and aseptic/sterile implements in the form of a single set. Particularly, to the best of our knowledge, no case has been found in the conventional arts wherein a kit for fat transplantation is constituted based on a numerically measurable concept under a standardized protocol. In fact, although current adipose tissue transplantation techniques exhibit considerable advancement over past adipose tissue transplantation techniques, there is still no significant suggestion of standardized protocols for adipose tissue transplantation or preparatory proceedings for sanitary adipose tissues. Further, there still remain a variety of problems to be solved; that is, currently available adipose tissue transplantation techniques suffer from disadvantages such as: necrosis and complications of transplanted tissues following adipose tissue transplantation; inconvenience and troubles associated with a need for complicated re-surgery due to reduction in the volume of transplanted adipose tissue after surgery; tissue fibrosis; and cell necrosis caused by physical damage.

### SUMMARY OF THE INVENTION

Therefore, the present invention has been made in view of the above problems, and it is an object of the present invention to 1) provide a kit for combined provision of easy manipulation for fat transplantation, and aseptic/sterile implements in the form of a single set concept, which until now has no precedent throughout the world, and 2) to supply such a kit to domestic and global medical markets, thereby securing upgraded technological capability in fat transplantation.

A second object of the present invention is to present a standardized protocol associated with use of a kit for autologous adipose tissue regeneration, which convenient and easy to manipulate.

A third object of the present invention is to minimize problems associated with the risk of infection that may occur upon performing fat transplantation, via aseptic and sterile treatment of all components; contained in the kit for adipose tissue transplantation.

A fourth object of the present invention is to induce regeneration of adipose tissues via use of a novel concept of biocompatible material, called Bio-Gel.

A fifth object of the present invention is to present new measures for solving the problems such as necrosis and complications of transplanted tissues, reduction in the volume of transplanted tissues after surgery and the like, via use of biocompatible Bio-Gel to supply nutrients of fat.

Consequently, a sixth object of the present invention is to provide a kit for adipose tissue regeneration and a method of using the same, which are suited for enhancing customer satisfaction by providing remarkably improved quality and reliability of the process.

In accordance with an aspect of the present invention, the above and other objects can be accomplished by the provision of a kit for adipose tissue regeneration constituted in the form of a single set kit, wherein the bottom of a kit box, equipped with a locking device, is provided with a washing container filled with an aseptic and sterile solution for washing adipose tissues, and a washing container insertion groove for containing the same; a first syringe container containing first syringes for washing and separating adipose tissues and injecting the wash solution and mixing of Bio-Gel, respectively, and a first syringe container insertion groove for containing the same; a second syringe container containing a number of second Bio-Gel syringes filled with biocompatible materials to promote adipogenesis induction and a second syringe container insertion groove for containing the same; connector containers containing connectors for use in injection of the wash solution and mixing of washed adipose tissues and biocompatible materials, and connector container insertion grooves for containing the same, and wherein the top of the kit box is provided with needle containers containing transplantation needles and needle container insertion grooves for containing the same; and a protocol in which standardization of fat transplantation is defined and a protocol insertion groove for containing the same.

In accordance with another aspect of the present invention, there is provided a method of using a kit for adipose tissue regeneration comprising: fill four 50 mL of the first syringes having pre-capped inlets for washing and separating adipose tissues, with 25 mL of adipose tissues harvested from donors, respectively; previously, fill four 50 mL of the first syringes for injection of the wash solution arranged in the kit with a solution for washing adipose tissues, then inject 20 mL of the adipose tissue-wash solution into the syringes filled with the 25 mL of adipose tissues, using a syringe connector, cap the inlet and manually and gently shake the syringes back and forth 10 to 25 times to wash the adipose tissues and centrifuge the washed adipose tissues so they will be densely concentrated (centrifuging step for centrifuging autologous adipose tissues). After centrifuging, open the cap of the syringe inlet to remove impurities below the adipose tissues, thereby obtaining pure adipose tissues (removing step for removal of impurities); repeat washing, centrifuging and impurity-removing steps; push a syringe containing completely pure, densely concentrated adipose tissues and a Bio-Gel arranged in the kit into an adipose tissue syringe using a syringe connector 60, push Bio-Gel thereto, draw a second Bio-Gel syringe, and homogeneously mix adipose tissues and Bio-Gel by pushing and pulling a 50 mL first syringe 30 for mixing Bio-Gel, 10 to 20 times. Upon mixing adipose tissues and Bio-Gel, one syringe filled with Bio-Gel is used for mixing per syringe containing 25 mL of adipose tissues (mixing step for mixing autologous adipose tissues and Bio-Gel); and inject the thus-mixed adipose tissues into a desired target region by a transplantation syringe (transplanting step for transplanting autologous adipose tissues).

### BRIEF DESCRIPTION OF THE DRAWINGS

FIG. 1 is a perspective view of a blank kit in which a multitude of various containers in accordance with the present invention were not included.

FIG. 2 is a perspective view of a kit in which a multitude of various containers applied to the present invention were included.

FIG. 3 is a cross-sectional view of a first syringe container applied to the present invention.

FIG. 4 is a cross-sectional view of a second syringe container applied to the present invention.

FIG. 5 is an exploded perspective view of a needle container and needle applied to the present invention.

FIG. 6 is an exploded perspective view of a connector container and connector applied to the present invention.

FIG. 7 is a view showing a state of a connector coupled between a first syringe and a second syringe, applied to the present invention.

### DETAILED DESCRIPTION OF THE PREFERRED EMBODIMENTS

The preferred embodiments of the present invention for accomplishing the above-mentioned objects will now be described in more detail with reference to the accompanying drawings.

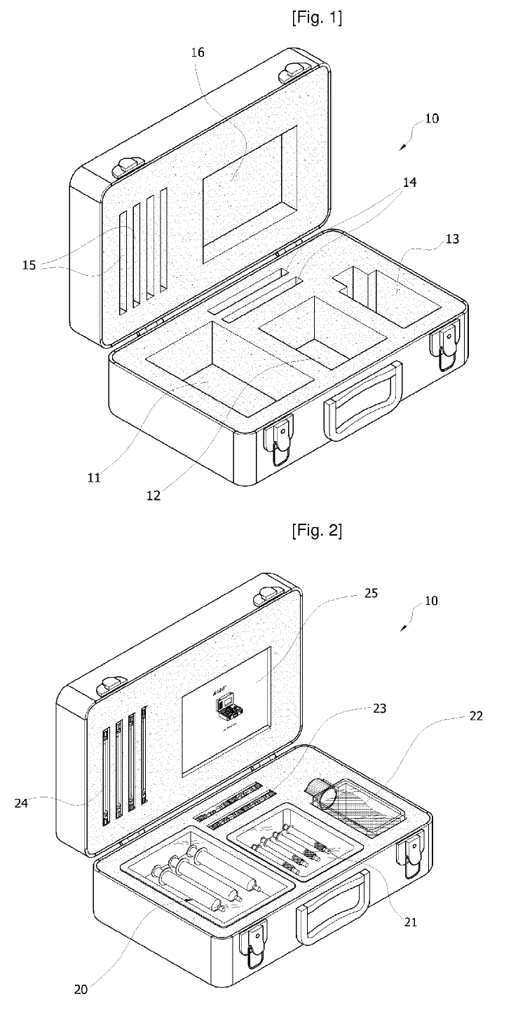

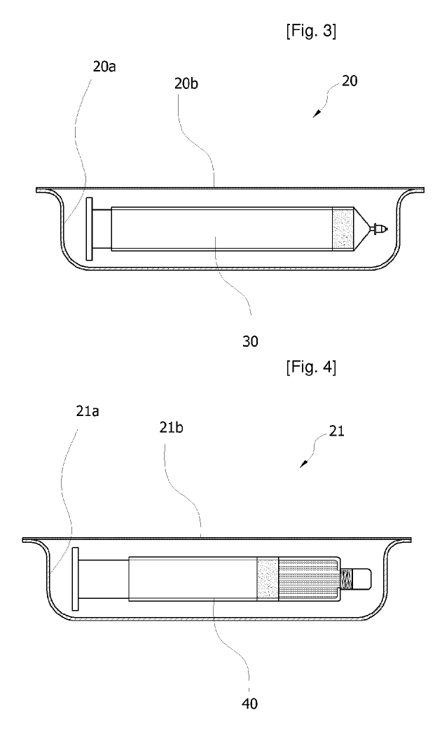

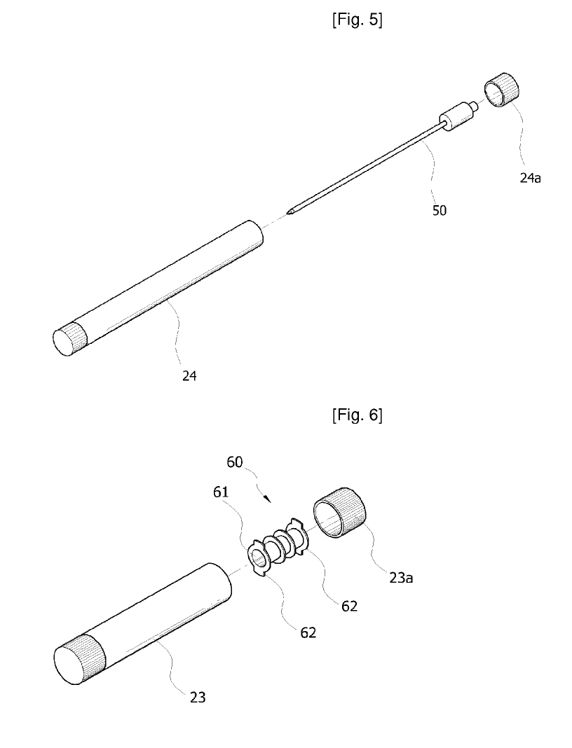

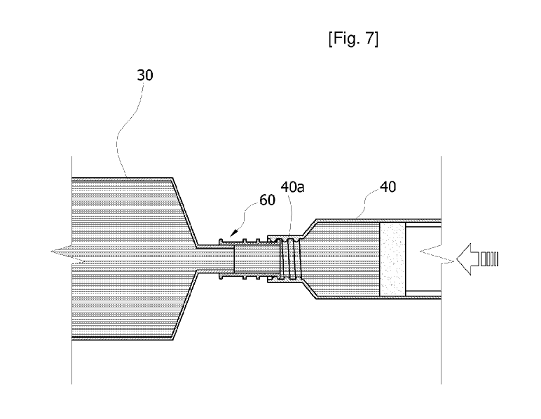

A kit for adipose tissue regeneration and a method of using the same, which are applied to the present invention, are constituted as shown in FIGS. 1 through 7.

In connection with description of the present invention hereinafter, if it is considered that specific description of known functions or constitution related to the present invention may make the subject matter of the present invention unclear, the detailed description thereof will be omitted.

Terms which will be described hereinafter are established taking into consideration functions in the present invention and may vary according to manufacturer's intention or general practices in the related art. Therefore, the terms used herein should be defined based on the contents of the specification of the present invention.

First, all of the kits used in the present invention can reconstruct 100 mL of adipose tissues per kit, and all components are disposable. Components of the kit are products that were sterilized in a space under germ-free conditions, and can be protected against the risk of infection upon performing adipose tissue transplantation.

In addition, in order to completely remove impurities contained in adipose tissues obtained from donors and at the same time, to enhance a maximum yield of mature adipocytes and preadipocytes in adipose tissues, there is needed a bottle which is filled with a solution for washing adipose tissues. Here, as the above-wash solution, “a semi-solid gel composition for adipose tissue regeneration” disclosed in Korean Patent Application No. 2005-0090202, which was filed by the present applicant, on Sep. 28, 2005, may be used. This composition is a mixture in which an adipose tissue washed in an adipose tissue-wash solution is in admixture with collagen, an adipogenesis-promoting solution, a serum and a medium.

Further, using the adipose tissue-wash solution of the present invention, it is possible to provide completely pure adipose tissues, which were obtained from donors, and thereby solve the problems associated with adipose tissue necrosis which may occur after adipose tissue transplantation.

In addition, in order to wash adipose tissues, a syringe for injection of the wash solution is previously filled with the adipose tissue-wash solution to achieve convenient washing. In order to wash adipose tissues contained in a syringe for washing and separation of adipose tissues, the wash solution contained in the syringe for injection of the wash solution can be conveniently injected in a specified volume by using a syringe connector. Further, the syringe connector is used to mix the washed adipose tissues with Bio-Gel constituted in the kit and mixing is handled with a syringe for mixing Bio-Gel.

In order to inject the mixed product of the adipose tissues and Bio-Gel into a target site for fat transplantation, a transplantation needle is necessary.

Further, in order to achieve objects of the present invention, the standardized protocol included in the kit is provided, and procedures of the protocol will be described hereinafter.

The kit of the present invention can be applied to various ranges including humans and animals, and target sites for adipose tissue transplantation may also include a broad area of soft tissue defects such as breast, wrinkles and soft tissue defect regions.

Hereinafter, technical constitution of the present invention will be described in more detail.

In the kit for adipose tissue regeneration according to the present invention, the bottom of a kit box equipped with a locking device is provided with a washing container 22 filled with an aseptic and sterile solution for washing adipose tissues and a washing container insertion groove 13 for containing the same, wherein the solution for washing adipose tissues is filtered and filled into a sterile washing container in an amount of 300 mL and the inlet of the container 22 is sealed again so as to prevent leakage of the wash solution.

In addition, at the bottom of the kit box, a first syringe container 20 containing a plurality of first syringes 30 for washing and separation of adipose tissues, injection of the wash solution and mixing of Bio-Gel, respectively and an first syringe container insertion groove 11 for containing the same are provided. Here, in order to conveniently wash large amounts of adipose tissues which were obtained from donors in a lump, and in order to remove impurities from those adipose tissues, 50 mL syringes are capped to prevent probable leakage of liquid, sterilized, and packaged into a set of six syringes.

At the bottom of the kit box is also provided a second syringe container 21 containing a plurality of second Bio-gel syringes 40 filled with biocompatible materials to promote adipogenesis induction and a second syringe container insertion groove 12 for containing the same. 2.5 mL of Bio-Gel for promoting adipogenesis induction and supplying nutrients of fat is filled in a 3 mL syringe which is then sterilized and packaged into a set of four syringes.

Further, connector containers 23 containing connectors 60 for use in injection of the wash solution and mixing of washed adipose tissues and biocompatible materials, and connector container insertion grooves 14 for containing the same, are provided at the bottom of the kit box. The syringe connectors, used for injection of the wash solution into another syringe or for convenient mixing of the washed adipose tissues with biocompatible materials, are sterilized and packaged into a set of four or five syringe connectors.

Meanwhile, the top of the kit box is provided with a plurality of needle containers 24 containing transplantation needles 50 and a plurality of needle container insertion grooves 15 for containing the same, according to size, respectively. In order to transplant the mixed adipose tissues into desired transplantation sites, transplantation needles corresponding to the respective target sites were respectively sterilized and packaged into a set of three to six needles.

Here, it is preferred to constitute the inside of the first and second syringe containers 20 and 21, needle containers 24 and connector containers 23 in an aseptic and sterile state.

In addition, on the inner circumference of one side of second Bio-Gel syringes 40, a thread part 40a is formed to be engaged with protrusions 62 formed on the outer circumference of the connector 60. The connector 60 is fabricated to have a hole 61 formed at the center thereof, such that the wash solution and the like pass through the hole 61.

Further, at the top of the kit box, a protocol 25 in which standardization of fat transplantation is defined and a protocol insertion groove 16 for containing the same are provided to kit 10 in the form of a single set.

Meanwhile, although the preferred embodiments of the present invention have been disclosed with reference to the accompanying drawings, those skilled in the art will recognize that the present invention may be embodied in different forms with various modifications.

It should be understood that the drawings and detailed description thereof are not intended to limit the invention to the particular form disclosed, but on the contrary, the intention is to cover all modifications, equivalents and alternatives falling within the spirit and scope of the invention as defined by the appended claims.

Effects of the a kit for adipose tissue regeneration and a method of using the same in accordance with the present invention, as constituted above, will be described hereinafter.

First, by using kit components that can be very easily and conveniently processed according to the standardized protocol and also are sterilized under germ-free conditions, while solving the problems exhibited by conventional adipose tissue transplantation techniques such as a low engraft rate and a body's absorption of transplanted adipose tissues and inducing adipose tissue regeneration, the present invention simultaneously provides solutions to problems of conventional fat transplantation, and convenient fat transplantation via use of a mixture of processed adipose tissues and adipogenesis-promoting Bio-Gel. The respective steps for this purpose will be described prior to introduction of the following examples.

That is, in accordance with the present invention, 25 mL of adipose tissues harvested from donors is filled in a total of four first syringes (50 mL) 30 having pre-capped inlets for washing and separating of adipose tissues, respectively.

Thereafter, an adipose tissue-wash solution is filled in the first 50 mL syringe 30 for injection of the wash solution arranged in the kit 10. Then 20 mL of the adipose tissue-wash solution is injected into a syringe filled with 25 mL of adipose tissues using a syringe connector 60, and while capping the inlet of the syringe, the syringe is gently shaken back and forth manually 10 to 25 times to wash the adipose tissues.

Next, the washed adipose tissues are centrifuged so as to be densely concentrated (centrifuging step for centrifuging autologous adipose tissues). Here, for centrifuging autologous adipose tissues, it is preferred to centrifuge at 1,200 to 3,000 rpm for five to 10 minutes after the washing step.

When centrifugation is carried out at less than 1,200 rpm for less than 5 min, impurities and the fat layer are not distinctively separated to some extent and adipose tissues are not densely concentrated to some extent. Therefore, centrifuging should be carried out at more than 1,200 rpm for more than five minutes. In contrast, when centrifuging is carried out at more than 3000 rpm for more than 10 minutes, separation between impurities and fat layer is distinctive, but preadipocytes present in adipose tissues may be disrupted. Therefore, it is preferred to perform centrifuging between 1,200 to 3,000 rpm for five to 10 min.

After centrifuging, the cap of the syringe inlet is opened to remove impurities below the adipose tissues, thereby obtaining pure adipose tissues (removing step, for removing impurities).

Additionally, washing, centrifuging and impurity removing steps are repeated. These steps are preferably repeated three or four times.

That is, when these steps are repeated fewer than three times, it is impossible to completely remove blood and other impurities. In contrast, when these steps are repeated more than four times, repeated washing and centrifuging may result in tissue necrosis. Therefore, it is preferred to repeat the above washing, centrifuging and impurity-removing steps three or four times.

Then, a syringe containing completely pure, densely concentrated adipose tissues and a Bio-Gel arranged in the kit are pushed into an adipose tissue syringe using a syringe connector 60, Bio-Gel is pushed thereto, a second Bio-Gel syringe 40 is drawn, a first syringe (50 mL) 30 for mixing Bio-Gel is then pushed and pulled 10 to 20 times to homogeneously mix adipose tissues and Bio-Gel. Here, mixing of adipose tissues and Bio-Gel is carried out in a manner that one syringe filled with Bio-Gel is used for mixing per syringe containing 25 mL of adipose tissues, thereby achieving mixing between autologous adipose tissues and Bio-Gel. As the Bio-Gel, one syringe filled with 2.0 to 3.0 mL of Bio-Gel is preferably used for mixing with adipose tissues.

That is, when the amount of Bio-Gel is less than 2.0 mL, homogeneity of adipose tissue regeneration may be deteriorated due to a low mixing ratio of Bio-Gel/adipose tissues. Therefore, the amount of Bio-Gel should be more than 2.0 mL. Conversely, when the amount of Bio-Gel is more than 3.0 mL, adipose tissue regeneration may be feasible, but biological properties of regenerated tissues (for example, elasticity) exhibit too high rigidity, which may result in sensation different from original properties intrinsic to gel and tissue fibrosis. Therefore, the amount of Bio-Gel should be less than 3.0 mL.

Finally, the mixed adipose tissues are injected into a desired target region by the corresponding transplantation syringe 50 (transplanting step for transplanting autologous adipose tissues). As discussed above, according to the present invention, it is possible to achieve easier and more convenient adipose tissue transplantation.

### EXAMPLE

According to the standardized protocol constituted within the kit for adipose tissue regeneration, all relevant procedures need to be performed. For this purpose, 100 mL of abdominal fat liposuctioned from a patient was put into four syringes for washing and separating of adipose tissues (25 mL/syringe). When adipose tissues were allocated into syringe, the inlet of the syringe was capped and therefore leakage of adipose tissues from the syringe was prevented upon manipulating the syringe. When adipose tissues were ready to be washed, 50 mL of a solution for washing adipose tissues constituted within the kit was filled in the syringe for injection of the wash solution. The cap of the inlet of the syringe containing adipose tissues to be washed was carefully open and connected to a syringe connector which was sterile packaged. Then, via the syringe connector from the syringe containing the wash solution, 20 mL of solution was transferred to each syringe which was then capped.

Then, the syringes were gently shaken back and forth 10 to 25 times such that the wash solution and adipose tissues were homogeneously mixed to wash adipose tissues. Whether adipose tissues were completely washed was confirmed by naked eyes, followed by centrifuging the respective syringes at 1,200 to 3,000 rpm for five to 10 min. After centrifuging was complete, the cap of the syringe inlet was open to remove impurities below the adipose tissue layer. These washing, centrifuging and impurity-removing steps were repeated thrice, thereby obtaining densely concentrated adipose tissues. Then, the densely concentrated adipose tissues were mixed with Bio-Gel which was filled with biocompatible materials inducing adipose tissue regeneration. Mixing with Bio-Gel was carried out by pushing Bio-Gel into the washed adipose tissue syringe, via connection with a syringe connector, replacing the Bio-Gel syringe with a 50 mL syringe for mixing Bio-Gel in the kit, and repeatedly pushing and pulling both syringes 10 to 20 times to thereby effect homogeneous mixing of adipose tissues and Bio-Gel. Upon mixing the washed adipose tissues and Bio-Gel, the syringe connector was disposed and was replaced one by one such that the risk of infection was minimized. Further, in principle, 2.5 mL of Bio-Gel is mixed per 25 mL of adipose tissues. After completion of mixing adipose tissues with Bio-Gel, four syringes each containing a total volume of 27.5 mL of mixture were prepared and used to reconstruct 100 mL of adipose tissue. After mixing, a size of syringe to be used was selected depending upon transplantation regions, and the mixture for adipose tissue regeneration was directly injected into the desired target region in a divided injection volume.

As apparent from the above description, the present invention provides a kit for combined provision of an easy manipulation method for fat transplantation and aseptic/sterile implements in the form of a single set concept, which has no precedent throughout the world hitherto, and supply such a kit to domestic and global medical market, thereby securing upgraded technological capability in fat transplantation. In particular, for this purpose, the present invention presents a standardized protocol associated with use of a kit for autologous adipose tissue regeneration, which is adapted to enable convenient and easy manipulation thereof. The present invention minimizes problems associated with the risk of infection that may occur upon performing fat transplantation, via aseptic and sterile treatment of all components arranged in a kit for adipose tissue transplantation. Further, the present invention enables induction of regeneration of adipose tissues via use of a novel concept of biocompatible material, called Bio-Gel. In addition, the present invention presents new measures for solving the problems such as necrosis and complications of transplanted tissues, reduction in the volume of transplanted tissues after surgery and the like, via use of biocompatible Bio-Gel to supply nutrients of fat. Consequently, the present invention provides a kit for adipose tissue regeneration and a method of using the same, which are suited for enhancing customer satisfaction via remarkably improved quality and reliability of the product.

What is claimed is:

1-7. (canceled)

8. A device for regenerating adipose tissue, which forms a set of a single kit (10), the device comprising:
a plurality of needle containers (24) containing transplantation needles (50) at a top of the kit, and insertion grooves (15) of the needle container for containing the tissues,
a protocol (25) having defined a standardization of fat transplantation and an insertion groove (16) of the protocol for containing the tissues,
a locking element equipped at a bottom of the kit,
a washing container (22) filled with an aseptic and sterile solution for washing adipose tissues and an insertion groove (13) of the washing container for containing the washed tissues;
a first syringe container (20) having a plurality of first syringes (30) for washing and separating the adipose tissues, injecting the wash solution and mixing a Bio-Gel, respectively, and the insertion groove (11) of the first syringe container for containing the washed tissues;
a second syringe container (21) having a plurality of second Bio-Gel syringes (40) filled with biocompatible materials to promote induction of adipogenesis and the insertion groove (12) of the second syringe container for containing the washed tissues; and
a plurality of connector containers (23) with containing connectors (60) using for injecting the wash solution and mixing the washed adipose tissues with biocompatible materials, and the insertion grooves (14) of the connector container for containing the washed tissues.

9. The device according to claim 8, wherein side first and second syringe containers (20, 21), needle containers (24) and connector containers (23) are in the aseptic and sterile state.

10. The device according to claim 8, wherein said second syringe (40) has formed an inner thread part (40a) to be engaged with protrusions (62) formed on the outer circumference of the connector (60), and the connector has a hole formed at the center thereof, such that the wash solution passes through the hole.

11. A method for regenerating adipose tissue by utilizing a regenerating kit, the method comprising steps of:
filling a total of four 50 mL of first syringes (30), having pre-capped inlets for washing and separating of adipose tissues, with 25 mL of adipose tissues harvested from a donor, respectively;
injecting a wash solution to the first 50 mL syringe (30) arranged in the kit (10) with a solution for washing adipose tissues, injecting 20 mL of an adipose tissue-wash solution into syringes filled with 25 mL of adipose tissues using a syringe connector (60), and then, while capping the inlet, manually and gently shaking the syringes back and forth 10 to 25 times to wash adipose tissues;
centrifuging the washed adipose tissues to cause them to be densely concentrated;
detaching the cap of syringe inlet briefly to remove impurities below the adipose tissues, thereby obtaining pure adipose tissues;
repeating the washing, centrifuging and impurity-removing steps;
pushing a syringe containing completely impurity-free, densely concentrated adipose tissues and a Bio-Gel arranged in the kit into an adipose tissue syringe using a syringe connector (60), pushing Bio-Gel thereto, drawing a second Bio-Gel syringe (40), and homogeneously mixing adipose tissues and Bio-Gel by pushing and pulling a first 50 mL syringe (30) for mixing Bio-Gel, 10 to 20 times. For mixing adipose tissues and Bio-Gel, one syringe filled with Bio-Gel is used for mixing per syringe containing 25 mL of adipose tissues; and
injecting the mixed adipose tissues into a desired target region using a transplantation syringe (50).

12. The method according to claim 11, wherein centrifuging autologous adipose tissues is carried out at 1,200 to 3,000 rpm for 5 to 10 min after the washing step.

13. The method according to claim 11, wherein the repeating step is carried out three or four times.

14. The method according to claim 11, wherein one syringe filled with 2.0 to 3.0 mL of Bio-Gel is used for mixing with adipose tissues, as the Bio-Gel.
